(19) United States
(12) Patent Application Publication
(10) Pub. No.: US 2005/0175651 A1
(43) Pub. Date: Aug. 11, 2005

(54) Aqueous dispersion of nanocapsules with an oily core

(75) Inventors: Jean-Thierry Simonnet, Cachan (FR); Pascal Richart, Paris (FR); Bruno Biatry, Vincennes (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/027,993

(22) Filed: Jan. 4, 2005

Related U.S. Application Data
(60) Provisional application No. 60/538,250, filed on Jan. 23, 2004.

(30) Foreign Application Priority Data
Jan. 9, 2004 (FR) 04 50057

(51) Int. Cl. A61K 7/135; A61K 9/16; A61K 9/50

(52) U.S. Cl. 424/401; 424/490

(57) ABSTRACT

The present invention relates to an aqueous dispersion of nanocapsules of core/shell type, in which: the core comprises at least one oil, the shell covering the core is of non-crosslinked polymeric nature and is water-insoluble and insoluble in the oil of the said core, the said nanocapsules having a mean size of less than or equal to 1 μm and having a degree of encapsulation of at least 8% by weight relative to the total weight of the said dispersion.

The present invention relates mainly to an aqueous dispersion of nanocapsules of core/shell type, to a process for preparing them and to their use especially in cosmetics and/or pharmaceuticals.

The field of the invention more particularly relates to the encapsulation of at least one oil, where appropriate combined with at least one liposoluble active material in a vector of nanocapsule type. Such vectors are particularly useful in cosmetics and pharmaceuticals since they ensure increased stability of the encapsulated active material and ensure its efficient vectorization to its site of action, which is generally the epidermis.

Many techniques have been proposed to date for preparing nanocapsules of this type.

For example, a certain number of nanocapsules are now produced by a technique known as “solvent overflow”. This technique is especially illustrated in document EP 274 961. It involves dissolution of the material to the encapsulated and of the polymer, which is to constitute the envelope of the nanocapsule, in a solvent. The formation of the nanocapsules is initiated by adding, with stirring, to this solution an intermediary solvent that is incapable of dissolving the polymer and the material to be encapsulated, but that is on the other hand miscible with the first solvent. Unfortunately, the degree of encapsulation obtained with this technique remains insufficient, since it generally does not exceed 8% by weight relative to the weight of the dispersion.

Patent application WO 01/64328 illustrates another technique, known as “phase inversion”, which involves the application to the emulsion under consideration of several cycles of raising and lowering the temperature to bring about this phase inversion. This technique is especially illustrated therein for the preparation of particles smaller than 100 nm, with an oily core coated with a wax or a lecithin, i.e. a core whose melting point may prove to be less than 20° C. This type of wall also has the drawback of being very permeable, sensitive to surfactants and chemically unstable since it is very sensitive to oxidation.

Patent U.S. Pat. No. 4,124,526 from the Monsanto Company itself proposes an alternative to this solvent overflow technique, which consists in obtaining particles by coacervation of a salt of a polycarboxylic polymer, by acidification of the suspension to a pH of between 5 and 8. This solvent-free process leads to particles of heterogeneous size, generally greater than one micrometre, and having high porosity.

Another route of access to nanocapsules of this type is based on the emulsification technique and involves the simultaneous synthesis of the polymer shell by crosslinking of the corresponding monomers.

For example, document FR 1 583 243 describes a process for manufacturing microcapsules containing oil droplets with a size ranging from 0.1 to several microns and which uses chemical crosslinking agents. This technique is also illustrated in document WO 02/09862.

Patent application WO 93/08908 uses this same technique to prepare nanocapsules whose shell is derived from the crosslinking of proteins.

Patent application WO 02/051536 itself describes suspensions of particles between 70 nm and 5 μm in size, obtained by crosslinking monomers, this crosslinking being initiated by exposing the suspension to UV irradiation.

As it turns out, this second route of access has the major drawback of requiring a crosslinking step that involves either chemical species of crosslinking agent type, which may pose problems in terms of harmlessness, or an external stimulus of UV irradiation type, which may be harmful to the biological active agents if they prove to be UV-sensitive.

Finally, patent application WO 99/43426 from Laboratoires Sérobiologiques describes lipid particles that are solid between 25 and 85° C., of between 10 nm and 5 mm in size, obtained by emulsification and without solvent. However, the particles described in the said document are particles necessarily consisting of at least one solid lipid of wax type and have no solid polymer coating, i.e. they are different from nanocapsules. When introduced into a composition containing surfactants, these particles show limited stability on account of the partial dissolution of the waxes by the surfactants, thus inducing the release of the encapsulated oily active materials.

In general, the processes described above are also dependent on the nature of the solvents used, which must be water-miscible. This imposes a large limitation in the choice of polymers that form the shell of the nanocapsule, which are by definition water-insoluble and lipophilic. Finally, these processes generally require very large volumes of solvent (water-miscible) and of water, which then need to be evaporated off in order finally only to obtain a dispersion containing not more than 8% by weight of degree of encapsulation.

Consequently, the dispersions of nanocapsules currently available are not found to be entirely satisfactory since they are not simultaneously satisfactory in terms of size, occasionally in terms of harmlessness, of stability and of general-purpose use regarding the nature of the active material to be encapsulated.

What is more, the content of encapsulated active material generally does not exceed 8% with nanocapsules of this type. However, to increase the performance of the encapsulated molecules, it would be necessary to have a degree of encapsulation that is, in any case, greater than 8% and in particular greater than 10%.

The objective of the present invention is specifically to propose nanocapsules that can satisfy in these respects, i.e. that are suitable for encapsulated in high proportion a large variety of oils and/or liposoluble active agents, which can advantageously have a size ranging from 40 nm to 150 nm, which are compatible with the use of a very large number of polymers and whose preparation requires smaller amounts of solvent than those of the conventional processes.

According to one of its aspects, the present invention relates to an aqueous dispersion of nanocapsules of core/shell type, in which:
the core comprises at least one oil,
the shell covering the core is of non-crosslinked polymer nature and is water-insoluble and insoluble in the oil of the said core, the said nanocapsules having a mean size of less than or equal to 1 μm and having a degree of encapsulation of at least 8% by weight relative to the total weight of the said dispersion.

According to another of its aspects, the present invention relates to a process for preparing an aqueous dispersion of nanocapsules of core/shell type, the core of which comprises at least one oil and the shell covering the core is of polymeric nature and is water-insoluble and insoluble in the oil(s) of the said core, comprising the steps consisting in:
a) preparing a one-phase liquid organic medium comprising at least:
one organic solvent, with a boiling point of less than or equal to 100° C.,
a polymer that is soluble in the said solvent medium,
an oil, where appropriate containing at least one liposoluble active material, and
a nonionic surfactant,
b) preparing an aqueous medium containing at least one nonionic surfactant and, where appropriate, an ionic surfactant,
c) dispersing the organic medium (a) in the aqueous medium (b) so as to obtain a pre-emulsion,
d) subjecting the pre-emulsion obtained in (c) to homogenization so as to obtain the formation of a dispersion of nanocapsules with a mean size of less than or equal to 1 μm,
e) evaporating off the organic solvent and, where appropriate, some of the water, and
f) recovering the said aqueous dispersion of nanocapsules thus obtained.

The process according to the invention is advantageous in several respects.

Firstly, as emerges from the text hereinbelow, it is found to be suitable for encapsulating a large diversity of plant, animal or synthetic oils.

The nanocapsules obtained according to this process may have a mean size of less than 150 nm, without this being harmful to the degree of encapsulation, which remains considerably higher than 10% by weight relative to the total weight of the aqueous dispersion.

Since preformed polymers are used, this makes it possible to effectively eliminate the problem of harmlessness more particularly posed by the use of monomers that involves that of at least one crosslinking agent.

Finally, the process of the invention is advantageously free of the two constraints posed by the nature of the solvents and by their volume of use with the processes discussed hereinabove.

Nanocapsules:

For the purposes of the present invention, the term “nanocapsules” denotes a structure of core-shell type, i.e. a structure consisting of a core forming or containing the material to be encapsulated, this core being enveloped with a continuous and water-insoluble protective shell. In other words, according to the invention, they are nanocapsules with a lipid core surrounded by a shell of polymeric nature.

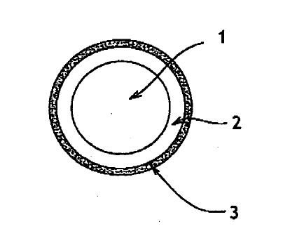

The nanocapsules according to the invention may be, for example, as shown diagrammatically in FIG. 1.

The nanocapsules shown in FIG. 1 comprise a core (1), comprising at least one oil, a shell (2) of polymeric nature, and, where appropriate, an outer lamellar phase (3).

The nanocapsules according to the invention differ especially from “nanospheres” consisting of a porous polymer matrix in which the active material is absorbed and/or adsorbed.

The nanocapsules according to the invention are advantageously less than or equal to 1 μm, in particular less than or equal to 500 nm and more particularly less than or equal to 250 nm in size. According to one particular embodiment, they are less than or equal to 150 nm, or even less than or equal to 125 nm, especially less than or equal to 100 nm and in particular less than or equal to 80 mn in size. The size is expressed as the medium intensity size supplied by a Brookhaven type BI 90 Plus granulometer, the measuring principle of which is based on quasi-elastic light scattering (QELS).

As mentioned previously, the dispersion according to the invention has a degree of encapsulation significantly higher than that of the conventional dispersions of nanocapsules as discussed previously.

This degree of encapsulation expressed as the weight of encapsulated material is at least 8% by weight relative to the total weight of the dispersion, in particular greater than or equal to 10% by weight, especially greater than or equal to 12.5% by weight, or even greater than or equal to 15% by weight, and more particularly greater than or equal to 17.5% by weight, relative to the total weight of the dispersion.

The shell of the nanocapsules is of non-polymeric, non-crosslinked nature, and is water-insoluble and insoluble in the oil of the core.

In general, any polymer, of natural or synthetic origin, which is soluble in a water-immiscible solvent, and especially those with a melting point lower than the boiling point of water at atmospheric pressure (100° C.), may be used according to the present invention.

These polymers may be biodegradable, for instance polyesters, or non-biodegradable.

As illustrations of polymers that are suitable for the invention, mention may especially be made of:
C₂-C₁₂ and in particular C₂-C₆ alkyl cyanoacrylate polymers, the alkyl radical possibly being chosen in particular from ethyl, n-butyl, hexyl, isobutyl and isohexyl radicals,
polymers formed from poly-L-lactides, poly-DL-lactides, polyglycolides and the corresponding copolymers, such as copoly(DL-lactides and glycolide)s, copoly(glycolides and caprolactones) and the like,
polycaprolactones, such as the polycaprolactones having a melting point ranging from 40 to 70° C. and a molecular weight of between 2000 and 100 000, such as for example those sold by the company Solvay under trade references CAPA 6806 (MW of 80 000), CAPA 6100 (MW of 10 000), CAPA 6506 (MW 50 000), CAPA 6250 (MW of 25 000), CAPA 2803 (MW of 8000) and CAPA 2403 D (MW of 4000),
3-hydroxybutyric acid polymers,
copolymers of vinyl chloride and of vinyl acetate, for example those sold under the name Rhodopas AX 8515® by the company Rhône-Poulenc,
copolymers of methacrylic acid and ester, especially of methacrylic acid and of methacrylic acid ester, for example those sold under the name Eudragit L 100® by the company Rohm Pharma,
polyvinyl acetophthalate,
cellulose acetophthalate,
poly vinylpyrrolidone/vinyl acetate copolymer,
poly(ethylene/vinyl acetate)s,
polyacrylonitriles,
polyacrylamides,
polyethylene glycols,
poly(C₁-C₄ hydroxyalkyl methacrylate)s and in particular poly(hydroxyethyl methacrylate)s,
cellulose derivatives such as cellulose esters of at least one C₁-C₄ carboxylic acid, especially mixed cellulose esters of two types of carboxylic acid,
polystyrene and copolymers of styrene and of maleic anhydride, copolymers of styrene and of acrylic acid, styrene/ethylene/butylene/styrene block terpolymers and styrene/ethylene/propylene/styrene block terpolymers,
styrene/alkyl alcohol oligomers,
terpolymers of ethylene, of vinyl acetate and of maleic anhydride,
polyamides,
polyethylenes,
polypropylenes,
organopolysiloxanes, including polydimethylsiloxanes,
poly(alkylene adipate)s, which include both homopolymers of adipic acid and of an alkanediol and copolymers of poly(ester ether) type, which may be linear or branched, obtained from adipic acid and from one or more alkanediols and/or ether diols and/or triols; the alkanediols used for the preparation of the said poly(alkylene adipate)s may be linear or branched C₂-C₆ alkanediols chosen from ethylene glycol, propylene glycol, 1,3-propanediol, 1,4-butanediol, 1,5-pentanediol, 1,6-hexanediol and neopentyl glycol. The ether diols are di-, tri- or tetra(C₂-C₄ alkylene) glycols such as diethylene glycol, triethylene glycol, tetraethylene glycol, dipropylene glycol, tripropylene glycol, tetrapropylene glycol, dibutylene glycol, tributylene glycol or tetrabutylene glycol; examples that may be mentioned more particularly include the Fomrez® products sold by the company Witco and the poly(ethylene) adipates from the company Scientific Polymer Products; examples that may also be mentioned include Eastar Bio® from Eastman Chemical (poly(tetramethylene adipate-co-terephthalate) and Ecoflex F BX 701® from BASF (1,4-butanediol/terephthalic acid/adipic acid terpolymer),
the polyester polyols obtained by polycondensation of an aliphatic dicarboxylic acid with at least two alkanediols or with at least one alkanediol and at least one hydroxyalkyl alkanediol, the aliphatic dicarboxylic acid possibly being adipic acid (1,6-hexanedioic acid); such polymers are described especially in document FR 2 836 381,
polysilsesquioxanie silicone polymers, especially a polyalkylsilsesquioxane of formula: (R-SiO3/2)ₓ₀ in which R represents a saturated or unsaturated, linear, branched or cyclic hydrocarbon-based radical; for example of the type —CₙH₂ₙ₊₁, with n being an integer ranging from 1 to 20, especially a methyl, ethyl, propyl, butyl, pentyl, hexyl, hexyl, octyl, nonyl, decyl, dodecyl, tridecyl, tetradecyl, hexadecyl, octadecyl or eicosyl radical; or alternatively an aryl group, especially phenyl or tolyl; a cycloalkyl group, especially cyclobutyl, cyclopentyl or cyclohexyl; an alkenyl group, especially vinyl or allyl; an aralkyl group, especially 2-phenylethyl or benzyl; R also possibly comprising one or more halogen atoms, especially fluorine or chlorine; preferably, R is a methyl, ethyl, propyl or phenyl radical; and x is the number of units, and may be between 1 and 10, especially 1 to 4; examples that may be mentioned include Belsil PMS MK® from Wacker and Resin KR 220® from Shin-Etsu,
dendritic polyesters containing a hydroxyl end function, especially those described in document FR 2 790 405,
polymers that are water-dispersible but soluble in water-immiscible solvents, for instance: polyesters, poly(ester amides), polyurethanes and vinyl copolymers bearing carboxylic acid and/or sulfonic acid functions, and in particular those described in document FR 2 787 729,
block copolymers that are insoluble in water at room temperature and solid at room temperature, containing at least one block of one of the above polymers, and
mixtures thereof.

These polymers or copolymers may have a weight-average molecular weight of between 1000 and 500 000 and in particular between 1500 and 100 000.

Poly(alkylene adipate)s, organopolysiloxanes, polycaprolactones, cellulose acetophthalate, cellulose acetobutyrate, cellulose esters, and polystyrene and derivatives thereof, are most particularly suitable for the invention.

Needless to say, a person skilled in the art is capable, on the basis of his knowledge, of adjusting the molecular weight of the selected polymer with respect to its concentration in the solvent in order to have a viscosity of the mixture that is compatible with satisfactory emulsification.

Encapsulable Active Material:

For the purposes of the present invention, the term “oil” denotes any natural plant or animal, or synthetic, oily substance that is liquid at and above 40° C., which has one or more proven biological activities and which is water-insoluble (less than 2% by weight at room temperature).

Non-limiting illustrations of these oils that may especially be mentioned include unsaturation-rich plant oils, for instance borage oil, fish oils, sunscreens, vitamins E, F and K and esters thereof, and mixtures thereof.

One or more liposoluble active materials that will be dissolved in a solvent oil may also be encapsulated. According to this variant, it is a mixture of at least two or even more components that is encapsulated. Examples of these liposoluble active principles that may be mentioned include vitamins such as vitamin A (retinol), vitamin D, carotenes, β-carotene and salicylic acid and derivatives thereof. For example, the salicylic acid derivatives that may be used are those described in documents FR-A-2 581 542, EP-A-378 936 and EP-A-570 230, and in particular 5-n-octanoylsalicylic acid, 5-n-decanoyl-salicylic acid, 5-n-dodecanoylsalicylic acid, 5-n-octylsalicylic acid, 5-n-heptyloxysalicylic acid and 4-n-heptyloxysalicylic acid.

Moreover, any molecule with a therapeutic effect used in the pharmaceutical field may also be encapsulated, provided that it is of oily nature and/or of sufficient solubility in the oil under consideration. These molecules may thus be steroidal or non-steroidal anti-inflammatory agents, antifingal agents, antibacterial agents, antibiotics, antimitotic agents, anaesthetics, analgesics, antiseptics and anti-viral agents.

However, they may also be liposoluble compounds that are active in other fields, for instance pesticides or herbicides.

Needless to say, the content of oil(s) and, where appropriate, of liposoluble active material(s) (oil or oil+active agent) dissolved in the solvent depends on the solubility of these ingredients.

In general, it is adjusted such that the degree of encapsulation can represent more than 8% and in particular from 10% to 40% of the total weight of the aqueous dispersion.

As mentioned previously, the nanocapsules according to the invention may be prepared by the process in accordance with the invention.

This process according to the invention especially involves, successively:
the dissolution of the oil, of the polymer(s) and, where appropriate, of the active material to be encapsulated in an organic solvent medium, which is generally non-aqueous,
the formation of an “organic solvent medium in an aqueous medium” pre-emulsion by dispersing this organic medium in an aqueous medium, and
the homogenization, generally by high pressure, of the pre-emulsion formed so as to form an aqueous dispersion of nanocapsules.

The organic solvent medium, or even some of the water, is then removed, generally by evaporation under reduced pressure, so as to obtain a dispersion of nanocapsules at the desired concentration.

According to one particular embodiment, the oil/polymer weight ratio used in the organic solvent medium is less than or equal to 30/1 and especially ranges from 1/25 to 25/1.

Organic Solvent Medium:

The organic solvent media under consideration according to the invention are at least partially water-immiscible. More specifically, their solubility in water should be less than 10% by weight in water at room temperature. Their boiling point is necessarily less than that of water, generally 100° C. at atmospheric pressure. As examples of solvents that are suitable for the invention, mention may be made in particular of ethyl acetate, butyl acetate, dichloromethane, cyclohexane, heptane, 1-chlorobutane, chloroform and ethyl formate, and mixtures thereof.

The proportion of organic solvent medium may be adjusted so as to constitute from 5% to 70% by weight relative to the total weight of the pre-emulsion obtained in step c).

Surfactants:

In order to facilitate the emulsification of the organic solvent medium incorporating the oil and, where appropriate, the active material to be encapsulated, and to control the stability of the corresponding emulsion, it is generally desirable to use at least one surfactant, especially a nonionic surfactant.

This surfactant or this mixture of surfactants is generally present at from 0.05% to 25% and especially from 1% to 20% by weight relative to the weight of the organic solvent medium to be dispersed. Its HLB value (Hydrophilic-Lipophilic Balance) is generally adjusted so as to be favourable to the formation of emulsions of oil-in-water type.

The following nonionic surfactants are especially suitable for the invention:
alkyl esters or ethers of glycerol or of polyglycerol consisting of from 1 to. 10 glycerol “units” and of at least one alkyl chain (acid for the esters and alcohol for the ethers) containing from 12 to 22 carbon atoms. It may be saturated or unsaturated, and branched or unbranched. Examples that may be mentioned include Nikkol DGMS® (diglyceryl monostearate), Nikkol decaglyn 2IS® (decaglyceryl duisostearate) and triglyceryl hexadecyl ether,
mixed esters of fatty acids or of fatty alcohols, of carboxylic acids and of glyceryl, chosen, for example, from mixed esters of C₈-C₂₂ fatty acid or fatty alcohol and of α-hydroxy acid and/or of succinic acid with glycerol, and mixtures thereof. By way of example, mention may be made of the mixed ester of glycerol and of the mixture of citric acid, lactic acid, linoleic acid and oleic acid (CTFA name: Glyceryl citrate/lactate/linoleate/oleate) sold by the company H\ddot{l}s under the name lmwitor 375®; the mixed ester of succinic acid and of isostearyl alcohol with glycerol (CTFA name: Isostearyl diglyceryl succinate) sold by the company Hüls under the name Imwitor 780 K®; the mixed ester of citric acid and of stearic acid with glycerol (CTFA name: Glyceryl stearyl citrate) sold by the company Hüls under the name Imwitor 370®; the mixed ester of lactic acid and of stearic acid with glycerol (CTFA name: Glyceryl stearate lactate) sold by the company Danisco under the name Lactodan B30® or Rylo LA30®,
ethoxylated fatty ethers or ethoxylated fatty esters comprising from 2 to 50 ethylene oxide units and at least one alkyl chain (acid for the esters and alcohol for the ethers) containing from 12 to 22 carbon atoms. It may be saturated or unsaturated, and branched or unbranched. Examples that will be mentioned include the Brij® series (ethoxylated fatty alcohols) sold by the company Uniqema, the Myrj® series (ethoxylated stearates) sold by the company Uniqema, and PEG 400 isostearate, also sold by Uniqema,
oxyethylenated or non-oxyethylenated fatty esters of sorbitan. They comprise at least one sorbitan unit and, when they are oxyethylenated, from 2 to 50 ethylene oxide units, and at least one alkyl chain (fatty acid) containing from 12 to 22 carbon atoms. It may be saturated or unsaturated, and branched or unbranched. Examples that will be mentioned include the Span® series (sorbitan esters) and the Tween® series (oxyethylenated sorbitan esters) sold by Uniqema,
sugar fatty esters or sugar fatty ethers. The surfactant used is chosen, for example, from C₈-C₂₂ fatty acid esters of sucrose, maltose, glucose and fructose, C₁₄-C₂₂ fatty acid esters of methylglucose, alkylpolyglucosides, and mixtures thereof The alkyl chain(s) may be saturated or unsaturated, and branched or unbranched.

The C₈-C₂₂ or C₁₄-C₂₂ fatty acids forming the fatty unit of the esters that may be used in the composition of the invention comprise at least one saturated or unsaturated linear alkyl chain, of 8 to 22 or of 14 to 22 carbon atoms, respectively. The fatty unit of the esters may be chosen especially from stearates, behenates, arachidonates, palmitates, myristates, laurates and caprates, and mixtures thereof. Stearates are especially used.

Examples of esters or mixtures of esters of fatty acid and of sucrose, maltose, glucose or fructose that may be mentioned include sucrose monostearate, sucrose distearate and sucrose tristearate, and mixtures thereof, such as the products sold by the company Croda under the name Crodesta® F50, F70, F110 and F160, respectively having an HLB (Hydrophilic-Lipophilic Balance) of 5, 7, 11 and 16; and an example of esters or mixtures of esters of fatty acid and of methylglucose that may be mentioned is methylglucose polyglyceryl-3 distearate, sold by the company Goldschmidt under the name Tegocare 450®. Mention may also be made of monoesters of glucose or of maltose such as methyl O-hexadecanoyl-6-D-glucoside and O-hexadecanoyl-6-D-maltoside.

The sugar fatty alcohol ethers that may be used as surfactants in the composition according to the invention may be chosen especially from the group comprising ethers or mixtures of ethers of C₈-C₂₂ fatty alcohol and of glucose, maltose, sucrose or fructose, and ethers or mixtures of ethers of C₁₄-C₂₂ fatty alcohol and of methylglucose. They are especially alkylpolyglucosides.

The C₈-C₂₂ or C₁₄-C₂₂ fatty alcohols forming the fatty unit of the ethers that may be used according to the invention comprise a saturated or unsaturated linear alkyl chain containing, respectively, from 8 to 22 or from 14 to 22 carbon atoms. The fatty unit of the ethers may be chosen especially from decyl, cetyl, behenyl, arachidyl, stearyl, palmityl, myristyl, lauryl, capryl and hexadecanoyl, and mixtures thereof such as cetearyl.

Examples of sugar fatty alcohol ethers that may be mentioned include alkylpolyglucosides such as decyl glucoside and lauryl glucoside sold, for example, by the company Henkel under the respective names Plantaren 2000® and Plantaren. 1200®, cetostearyl glucoside optionally as a mixture with cetostearyl alcohol, sold, for example, under the name Montanov 68® by the company SEPPIC, under the name Tegocare CG90® by the company Goldschmidt and under the name Emulgade KE3302® by the company Henkel, and also arachidyl glucoside, for example in the form of the mixture of arachidyl and behenyl alcohols and of arachidyl glucoside, sold under the name Montanov 202® by the company SEPPIC,
Block copolymers of ethylene oxide and of propylene oxide. The block copolymers of ethylene oxide and of propylene oxide that may be used as surfactants in the compositions according to the invention may be chosen especially from the block copolymers of formula (A):
   HO(C₂H₄O)ₓ(C₃H₆O)_{y}(C₂H₄O)_{z}H (A)
   x, y and z are integers such that x+z may range from 2 to 280 and may preferably range from 14 to 100. These polymers are sold especially under the name Pluronic® or Lutrol® by BASF, or Symperonic® by Uniqema,
hydrogenated or non-hydrogenated soybean or egg lecithins, optionally enriched in phosphatidylcholine,
silicone surfactants comprising at least one oxyethylene or oxypropylene chain. Examples that may be mentioned include those described in patents U.S. Pat. No. 5,364,633 and U.S. Pat. No. 5,411,744, and in particular a compound of formula (I): 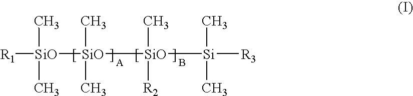
   in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the condition that A and B are not simultaneously equal to zero;
x is an integer ranging from 1 to 6;
y is an integer ranging from 1 to 30;
z is an integer ranging from 0 to 5.

According to one particular embodiment, in the compound of formula (I), the alkyl radical is a methyl radical, x is an integer ranging from 2 to 6 and y is an integer ranging from 4 to 30.

Examples of silicone surfactants of formula (I) that may be mentioned include the compounds of formula (II): 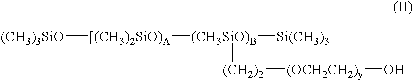
in which A is an integer ranging from 20 to 105, B is an integer ranging from 2 to 10 and y is an integer ranging from 10 to 20.

Examples of silicone surfactants of formula (I) that may also be mentioned include the compounds of formula (III):
HO—(CH₂CH₂O)_{y}—(CH₂)₃—[(CH₃)₂SiO]_{A′}—(CH₂)₃—(OCH₂CH₂)_{y}—OH (III)
in which A′ and y are integers ranging from 10 to 20.

Compounds of the invention that may be used include those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; A is 27, B is 3 and y is 12. The compound Q4-3667 is a compound of formula (III) in which A is 15 and y is 13. But also:
alkyl ether citrates,
alkoxylated alkenyl succinates,
alkoxylated glucose alkenyl succinates,
alkoxylated methyl glucose alkenyl succinates.

These surfactants may be used alone or in combination. Their content, relative to the encapsulated oily phase (after evaporating off the solvent) may be between 0.1% and 30% by weight.

In order to improve the stability of this emulsion, or even to slightly further reduce the size of the drops of the organic solvent medium, it may be advantageous to add from 0.01% to 5% by weight, relative to the total weight of the dispersion, of at least one water-soluble ionic surfactant with an HLB value of greater than 11. This type of ionic surfactant appears to generate an electric charge that the surface of the nanocapsules and thus promotes the manifestation of electrostatic repulsions between them. This or these ionic, anionic or cationic surfactant(s) may be chosen from:
anionic amphiphilic lipids, for instance:
   alkali metal salts of dicetyl and dimyristyl phosphate,
   alkali metal salts of cholesteryl sulfate,
   alkali metal salts of cholesteryl phosphate,
   lipoamino acids and salts thereof, such as monosodium and disodium acylglutamates, for instance the disodium salt of N-stearoyl-L-glutamic acid sold under the name Amisoft HS21P by the company Ajinomoto,
   the sodium salts of phosphatidic acid,
   phospholipids,
   alkylsulfonic derivatives especially of formula: 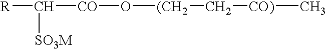
      in which R represents C₁₆-C₂₂ alkyl radicals, in particular C₁6H₃₃ and C₁₈H₃₇ radicals, taken as a mixture or separately, and M is an alkali metal or alkaline-earth metal such as sodium,
cationic amphiphilic lipids of quaternary ammonium salt type, and fatty amines and salts thereof, for instance:
   the quaternary ammonium salts of general formula (IV) below: 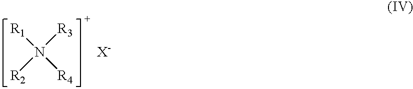
      in which the radicals R₁, R₂, R₃ and R₄, which may be identical or different, represent a linear or branched aliphatic radical containing from 1 to 30 carbon atoms or an aromatic radical such as aryl or alkylaryl. The aliphatic radicals may comprise hetero atoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl, containing from about 1 to 30 carbon atoms; X is an anion chosen from the group of halides, phosphates, acetates, lactates, (C₂-C₆)alkyl sulfates, and alkyl- or alkylarylsulfonates. Quaternary ammonium salts of formula (IV) that are preferred include, firstly, tetraalkylammonium chlorides, for instance dialkyldimethylammonium or alkyltrimethylammonium chlorides, in which the alkyl radical contains from about 12 to 22 carbon atoms, in particular behenyltrimethylammonium, distearyldimethylammonium, cetyltrimethylammonium or benzyldimethylstearylammonium chloride, or alternatively, secondly, stearamidopropyldimethyl(myristyl acetate)ammonium chloride sold under the name “Ceraphyl 70®” by the company Van Dyk,
Quaternary ammonium salts of imidazolinium, for instance those of formula (V) below: 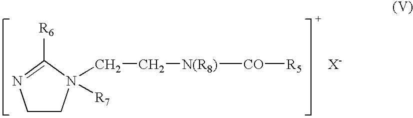
   in which R₅ represents an alkenyl or alkyl radical containing from 8 to 30 carbon atoms, for example fatty acid derivatives of tallow; R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R₈ represents a hydrogen atom or an alkyl radical containing from 1 to 4 carbon atoms; X is an anion chosen from the group of halides, phosphates, acetates, lactates, alkyl sulfates, and alkyl- or alkylarylsulfonates. Preferably, R₅ and R₆ denote a mixture of alkenyl or alkyl radicals containing from 12 to 21 carbon atoms, for example fatty acid derivatives of tallow, R₇ denotes a methyl radical, R₈ denotes hydrogen. Such a product is sold, for example, under the name “Rewoquat W 75” by the company Rewo,
Diquaternary ammonium salts of formula (VI) below: 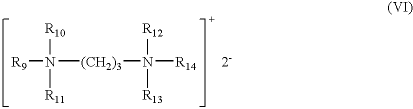
   in which R₉ denotes an aliphatic radical containing from about 16 to 30 carbon atoms; R₁₀, R₁₁, R₁₂, R₁₃ and R₁₄ are chosen from hydrogen and an alkyl radical containing from 1 to 4 carbon atoms; and X is an anion chosen from the group of halides, acetates, phosphates, nitrates and methyl sulfates. Such diquaternary ammonium salts especially include propane tallow diammonium dichloride.

The content of ionic surfactant, when it is combined with the nonionic surfactant(s) present in the aqueous medium, is generally adjusted so as to represent from 2% to 100% by weight relative to the weight of the medium.

Step c) is performed by dispersing the organic solvent medium (a) in the aqueous medium (b). This dispersion is prepared under conditions such that a pre-emulsion is obtained.

For the purposes of the present invention, this pre-ernulsion qualifies a state in which the organic medium (a) is present in the form of droplets ranging from 500 nm to 10 μm and in particular from 1 to 5 μm in size in the aqueous medium constituting the continuous phase. The size of the droplets may especially be controlled using a light-scattering laser granulometer.

The formation of the dispersion of the expected nanocapsules is performed during step (d) by subjecting this pre-emulsion to stiring with a shear effect, for example at at least 10 000 rpm, or agitation with a cavitation effect. According to one particular embodiment, this stirring is obtained by homogenizing the pre-emulsion in a high-pressure homogenizer, generally at a pressure of from 50 to 1500 bar, in particular from 200 to 1200 bar and especially greater than 400 bar. This shear effect may also be obtained using an ultrasound device such as the Sonitube from the company Sodeva.

The organic solvent medium contained in this emulsion is then removed, generally by evaporation according to standard techniques, for example using a rotary evaporator under reduced pressure. This evaporation may be performed until a fraction of the aqueous phase has evaporated off, which is indication of the total removal of the organic solvent medium.

After this step, a dispersion of nanocapsules in accordance with the invention is obtained.

A subject of the present invention is also the use of a dispersion of nanocapsules in accordance with the invention for the preparation of a cosmetic, dermatological or pharmaceutical composition.

The nanocapsules according to the invention may be introduced into any type of liquid, semi-solid or even solid galenical formulation. They may especially be gels, oil/water, water/oil or multiple (W/O/W or O/W/O) emulsions, sera or lotions. It is possible, for example, to introduce from 0.5% to 60% of an aqueous dispersion of nanocapsules in accordance with the invention into a cosmetic, dermatological or pharmaceutical composition.

Another aspect of the invention relates to a cosmetic, dermatological or pharmaceutical composition comprising, in a physiologically acceptable medium, at least one aqueous dispersion of nanocapsules according to the invention.

The formulations described containing nanocapsules may especially be intended for caring for and/or making up the hair, the skin and the nails.

Moreover, the nanocapsules of the present invention may be used for pharmaceutical purposes for the oral, peritoneal, intravenous, intramuscular or ophthalmic vectorization of medicinal products.

The examples given below are presented as non-limiting illustrations of the field of the invention.

All of the aqueous dispersions given in the examples below were prepared according to the following protocol:

A pre-emulsion of organic solvent medium/water type is prepared using an Ultra-Turrax mixer (IKA) for 5 minutes at 10 000 rpm, and is then homogenized using a Soavi OBL 20 high-pressure homogenizer, twice at 500 bar so as to obtain the expected dispersion of nanocapsules. The solvent is removed under reduced pressure using a rotary evaporator and a certain amount of water.

### EXAMPLE 1

Preparation of Macadamia Oil Nanocapsules

| Liquid solvent medium | | |
|---|---|---|
| Macadamia oil | 25.0 | g |
| Soybean lecithin | 5.0 | g |
| Isophthalic polyester (AQ38S from | 5.0 | g |
| Eastman Chemical) | | |
| Dichloromethane | 125 | ml |
| Aqueous medium | | |
| Distilled water | 500 | ml |
| Poloxamer 338 (sold by BASF) | 2.5 | g |

In this example, after evaporating off the dichloromethane, 115 nm nanocapsules are obtained, which are perfectly stable after 2 months at 45° C. The final aqueous dispersion contains 5% by weight of oil and 1% by weight of polymer, relative to the total weight of the dispersion.

### EXAMPLE 2

Preparation of Cottonseed Oil Nanocapsules

| | | |
|---|---|---|
| Organic solvent medium | | |
| Cottonseed oil | 37.5 | g |
| Belsil PMS MK (Wacker) | 3.0 | g |
| Dimethicone copolyol (DC 2-5695 | 6.0 | g |
| from Dow Corning) | | |
| Dichloromethane | 250 | ml |
| Aqueous medium | | |
| Distilled water | 375 | ml |
| Disodium N-Stearoyl-L-Glutamic | 3.0 | g |
| acid (Amisoft HS21P from Ajinomoto) | | |

According to the procedure described above, a dispersion of nanocapsules with a mean size of 98 nm dispersed in 300 ml of water is obtained. The aqueous dispersion contains 12.5% by weight of cottonseed oil and 1% by weight of polymer. These capsules are perfectly stable for 2 months at 45° C.

### EXAMPLE 3

Preparation of Vitamin E Nanocapsules

| | | |
|---|---|---|
| Organic solvent medium | | |
| Vitamin E acetate | 75.0 | g |
| Cellulose acetobutyrate (CAB-381-0.5 Eastman) | 11.25 | g |
| Dimethicone Copolyol (DC2-5695 from | 3.75 | g |
| Dow Corning) | | |
| Dichloromethane | 250 | ml |
| Aqueous medium | | |
| Distilled water | 375 | ml |
| Disodium N-Stearoyl-L-Glutamic acid | 1.875 | g |
| (Amisoft HS21P from Ajinomoto) | | |

After evaporating off the dichloromethane, a dispersion of nanocapsules with a mean size of 124 nm that are perfectly stable for 2 months at 45° C. is obtained. The aqueous dispersion contains 20% by weight of oil and 3% by weight of polymer.

### EXAMPLE 4

Preparation of Isocetyl Palmitate Nanocapsules

| | | |
|---|---|---|
| Organic solvent medium | | |
| Isocetyl palmitate | 56.25 | g |
| Polycaprolactone (CAPA6100 from Solvay) | 7.5 | g |
| Dimethicone Copolyol (DC2-5695 from | 3.75 | g |
| Dow Corning) | | |
| Dichloromethane | 250 | ml |
| Aqueous medium | | |
| Distilled water | 375 | ml |
| Disodium N-Stearoyl-L-Glutamic acid | 1.875 | g |
| (Amisoft HS21P from Ajinomoto) | | |

After evaporating off the dichloromethane, a dispersion of nanocapsules with a mean size of 103 nm that are perfectly stable for 2 months at 45° C. is obtained. The aqueous dispersion contains 15% by weight of oil and 2% by weight of polymer.

### EXAMPLE 5

Preparation of Vitamin E Nanocapsules

| | | |
|---|---|---|
| Organic solvent medium | | |
| Vitamin E acetate | 75.0 | g |
| Eastar Bio ® from Eastman Chemical | 3.75 | g |
| (Poly(tetramethylene Adipate-co-terephthalate) | | |
| Dimethicone Copolyol (DC2-5695 from | 3.75 | g |
| Dow Corning) | | |
| Dichloromethane | 250 | ml |
| Aqueous medium | | |
| Distilled water | 375 | ml |
| Disodium N-Stearoyl-L-Glutamic acid | 1.875 | g |
| (Amisoft HS21P from Ajinomoto) | | |

After evaporating off the dichloromethane, an aqueous dispersion of nanocapsules with a mean size of 240 nm that are perfectly stable after 2 months at 45° C. is obtained. The aqueous dispersion contains 20% by weight of oil and 1% by weight of polymer.

1. Aqueous dispersion of nanocapsules of core/shell type, in which:
the core comprises at least one oil,
the shell covering the core is of non-crosslinked polymer nature and is water-insoluble and insoluble in the oil of the said core, the said nanocapsules having a mean size of less than or equal to 1 μm and having a degree of encapsulation of at least 8% by weight relative to the total weight of the said dispersion.

2. Aqueous dispersion according to claim 1, characterized in that the nanocapsules have a mean size of less than or equal to 500 nm, especially less than or equal to 250 nm, or even less than or equal to 150 nm, and more particularly less than or equal to 100 nm.

3. Aqueous dispersion according to claim 1, characterized in that the degree of encapsulation is greater than or equal to 10% by weight, and especially greater than or equal to 12.5% by weight, or even greater than or equal to 15% by weight, and more particularly greater than or equal to 17.5% by weight relative to the total weight of the dispersion.

4. Aqueous dispersion according to claim 1, characterized in that the polymer of the shell of the nanocapsules has a weight-average molecular weight ranging from 1000 to 500 000 and especially from 1500 to 100 000.

5. Aqueous dispersion according to 4 claim 1, characterized in that the polymer of the shell of the nanocapsules has a melting point of less than 100° C.

6. Aqueous dispersion according to claim 1, characterized in that the shell of the nanocapsules comprises at least one polymer chosen from:
C₂-C₁₂ alkyl cyanoacrylate polymers,
poly-L-lactides, poly-DL-lactides, polyglycolides and the corresponding copolymers,
polycaprolactones,
3-hydroxybutyric acid polymers,
copolymers of vinyl chloride and of vinyl acetate,
polyvinyl acetophthalate, cellulose acetophthalate,
poly vinylpyrrolidone/vinyl acetate copolymer,
poly(ethylene/vinyl acetate)s,
copolymers of methacrylic acid and ester,
polyacrylonitriles,
polyacrylamides,
polyethylene glycols,
poly(C₁-C₄ hydroxyalkyl methacrylate)s,
cellulose derivatives,
polystyrene and copolymers thereof,
styrene/alkyl alcohol oligomers,
terpolymers of ethylene, of vinyl acetate and of maleic anhydride,
polyamides
polyethylenes,
polypropylenes,
organopolysiloxanes,
poly(alkylene adipate)s,
polyester polyols,
polysilsesquioxane silicone polymers,
dendritic polyesters containing a hydroxyl end function, and
mixtures thereof.

7. Aqueous dispersion according to claim 1, characterized in that the polymer of the shell of the nanocapsules is chosen from:
polycaprolactones,
polyvinyl acetophthalate, cellulose acetophthalate,
copolymers of methacrylic acid and ester,
cellulose derivatives,
polystyrene and copolymers thereof,
polyamides,
organopolysiloxanes,
poly(alkylene adipate)s, and
mixtures thereof.

8. Aqueous dispersion according to claim 1, characterized in that the oil(s) present in the core of the nanocapsules is liquid at and above a temperature of 40° C.

9. Aqueous dispersion according to claim 1, characterized in that the oil encapsulated in the core of the said nanocapsules is chosen from unsaturation-rich plant oils, especially borage oil, fish oils, sunscreens, vitamins E, F and K and esters thereof, and mixtures thereof.

10. Aqueous dispersion according to claim 1, characterized in that the oil encapsulated in the nanocapsules also comprises at least one liposoluble active material.

11. Aqueous dispersion according to claim 10, characterized in that the said liposoluble active material is chosen from antibiotics, antifungal agents, anaesthetics, analgesics, antiseptic agents, anti-viral agents, pesticides and herbicides, and mixtures thereof.

12. Aqueous dispersion according to claim 10, characterized in that the said liposoluble active material is chosen from vitamins, especially vitamin A, vitamin D, carotenes, β-carotene and salicylic acid, and derivatives thereof.

13. Process for preparing an aqueous dispersion of nanocapsules of core/shell type, the core of which comprises at least one oil and the shell covering the core is of water-insoluble polymeric nature and is insoluble in the oil(s) of the said core, comprising the steps consisting in:
a) preparing a one-phase liquid organic medium comprising at least:
one organic solvent, with a boiling point of less than 100° C.,
a polymer that is soluble in the said solvent medium,
an oil, where appropriate containing at least one liposoluble active material, and
a nonionic surfactant,
b) preparing an aqueous medium containing at least one nonionic surfactant and, where appropriate, an ionic surfactant,
c) dispersing the organic medium (a) in the aqueous medium (b) so as to obtain a pre-emulsion,
d) subjecting the pre-emulsion obtained in (c) to homogenization so as to obtain the formation of a dispersion of nanocapsules with a mean size of less than or equal to 1 μm,
e) evaporating off the organic solvent, and
f) recovering the said aqueous dispersion of nanocapsules thus obtained.

14. Process according to claim 13, characterized in that the nanocapsules obtained in step d) are less than or equal to 500 nm, especially less than or equal to 250 nm and in particular less than or equal to 150 nm in size.

15. Process according to claim 13, characterized in that the aqueous dispersion obtained after step e) has a degree of encapsulation of greater than or equal to 8% by weight, especially greater than or equal to 10% by weight, in particular greater than or equal to 12.5% by weight, or even greater than or equal to 15% by weight, relative to the total weight of the aqueous dispersion.

16. Process according to claim 13, characterized in that the oil/polymer weight ratio of step a) is less than or equal to 30/1 and especially ranges from 1/25 to 25/1.

17. Process according to claim 13, characterized in that the proportion of organic solvent medium used in step a) is adjusted so as to constitute from 5% to 70% by weight relative to the total weight of the pre-emulsion obtained after step c).

18. Process according to claim 13, characterized in that the solvent is chosen from ethyl acetate, butyl acetate, dichloromethane, cyclohexane, heptane, 1-chlorobutane, chloroform and ethyl formate, and mixtures thereof.

19. Process according to claim 13, characterized in that the polymer is of the shell of the nanocapsules has a melting point of less then 100° C.

20. Process according to claim 13, characterized in that the oil is present in the core of the nanacapsules is liquid at and above a temperature of 40° C..

21. Process according to claim 13, characterized in that the liposoluble active material chosen from vitamins, especially vitamin A, vitamin D, carotenes, β-carotene and salicylic acid, and derivatives..

22. Process according to claim 13, characterized in that the nonionic surfactant of steps a) and b) is present in a content ranging from 0.05% to 25% by weight and especially from 1% to 20% by weight relative to the weight of the organic solvent medium.

23. Process according to claim 13, characterized in that the nonionic surfactant of steps a) and b) is chosen from:
alkyl esters or ethers of glycerol or of polyglycerol consisting of from 1 to 10 glycerol “units” and of at least one alkyl chain (acid for the esters and alcohol for the ethers) containing from 12 to 22 carbon atoms,
mixed esters of fatty acids or of fatty alcohols, of carboxylic acid and of glycerol,
ethoxylated fatty ethers or ethoxylated fatty esters comprising from 2 to 50 ethylene oxide units and at least one alkyl chain (acid for the esters and alcohol for the ethers) containing from 12 to 22 carbon atoms,
oxyethlenated or non-oxyethylenated fatty esters of sorbitan. They comprise at least one sorbitan unit and, when they are oxyethylenated, from 2 to 50 ethylene oxide units, and at least one alkyl chain (fatty acid) containing from 12 to 22 carbon atoms,
sugar fatty esters or sugar fatty ethers,
block copolymers of ethylene oxide and of propylene oxide,
hydrogenated or non-hydrogenated soybean or egg lecithins, optionally enriched in phosphatidylcholine,
silicone surfactants comprising at least one oxyethylene and/or oxypropylene chain, and
mixtures thereof.

24. Process according to claim 13, characterized in that the aqueous medium of step b) comprises at least one ionic surfactant present in a proportion of from 2% to 100% by weight relative to the weight of nonionic surfactant.

25. Process according to claim 13, characterized in that the said ionic surfactant of step b) is chosen from:
alkali metal salts of dicetyl and dimyristyl phosphate,
alkali metal salts of cholesteryl sulfate,
alkali metal salts of cholesteryl phosphate,
lipoamino acids and salts thereof,
the sodium salts of phosphatidic acid,
phospholipids,
alkylsulfonic derivatives,
quaternary ammonium salts,
the quaternary ammonium salts of imidazolinium,
diquatemary ammonium salts of formnula (VI) below: 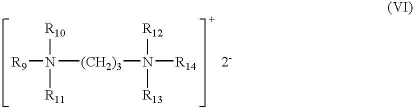
in which R₉ denotes an aliphatic radical containing from about 16 to 30 carbon atoms; R₁₀, R₁₁, R₁₂, R₁₃ and R₁₄ are chosen from hydrogen and an alkyl radical containing from 1 to 4 carbon atoms; and X is an anion chosen from the group of halides, acetates, phosphates, nitrates and methyl sulfates, and
mixtures thereof.

26. Process according to claim 13, characterized in that the homogenization is performed using a high-pressure homogenizer, especially at a pressure ranging from 50 to 1500 bar and in particular ranging from 200 to 1200 bar, or by ultrasound.

27. Dispersion of nanocapsules obtained by the process defined according to claim 13.

28. Use of an aqueous dispersion of nanocapsules according to claim 1, for the preparation of a cosmetic, dermatological or pharmaceutical composition.

29. Cosmetic, dermatological or pharmaceutical composition comprising, in a physiologically acceptable medium, at least one aqueous dispersion as defined according to claim 1.
